(19) United States
(12) Patent Application Publication
(10) Pub. No.: US 2018/0116345 A1
(43) Pub. Date: May 3, 2018

(54) SURFACE FASTENER FEMALE MATERIAL, SURFACE FASTENER, AND ABSORBENT GOOD

(71) Applicant: 3M INNOVATIVE PROPERTIES COMPANY, St. Paul, MN (US)

(72) Inventor: Kenichiro Morishita, Tokyo (JP)

(21) Appl. No.: 15/565,432

(22) PCT Filed: Apr. 8, 2016
(86) PCT No.: PCT/US2016/026549

§ 371 Date: Oct. 10, 2017

(30) Foreign Application Priority Data
Apr. 15, 2015 (JP) 2015-083773

(51) Int. Cl. A44B 18/00; D04B 21/02; A61F 13/62

(52) U.S. Cl. CPC A44B 18/0034; D10B 2501/0632; A61F 13/622; D04B 21/02

(57) ABSTRACT

Problem: To provide a surface fastener female material with a favorable print outer appearance without using a sublayer such as a print layer support film or the like, with excellent engagement properties and durability. Solution: A surface fastener female material containing a knitted fabric, wherein the knitted fabric is woven from a back yarn, a middle yarn, and a front yarn; the back yarn, middle yarn, and front yarn alternatingly form a joining point (I) and a joining point (II); the front yarn forms a close stitch at joining point (I), joining point (II), or both of these, and the back yarn contains at least 50% treated yarn by the total thread count standard.

### FIELD OF THE INVENTION

The present disclosure relates to a surface fastener female material, a surface fastener, and an absorbent good.

### BACKGROUND ART

Conventionally, surface fasteners have been widely used for fixing, binding, and the like of a variety of goods such as fiber products, plastic products, paper products, industrial parts, electronic parts, and building materials; for example, absorbent goods (such as diapers and the like) with a surface fastener as the engagement material are known. As for a surface fastener, surface fasteners with a variety of engagement designs are known, such as, for example, a pair consisting of a male material having a hook-shaped engagement element and a female material having a loop-shaped engagement element and the like.

Patent Document 1 describes a knit provided with a weft insert knit that is a weft insert knit used as a female knit in a hook-and-loop fastener, having a front side and a back side, wherein the back side has a plurality of stitch wheels separated from each other, wherein the loop part of the stitch protrudes outward and is connected to the wheel at base only, and neighboring loops on each wheel form free loops that slant sideways alternating in a suitable direction, and a weft inserted on the side of the knit between the front side and the back side of the knit and extending so as to cut across the entire width of the knit.

Patent Document 2 describes a loop material containing continuous multi-filament textured yarn joined intermittently to the foundation wherein the yarn is less than 50 deniers, and has a breaking energy of at least 60 Joules/g and stretches less than 45%.

Patent Document 3 describes an interwoven fabric used as a loop member of a hook and loop closed system including a base weave formed from base yarn and a pile weave formed from pile yarn, where the pile yarn is at least 7 grams/denier and is a strong monofilament yarn of from about 50 to 200 deniers with height 2.0 to 3.0 millimeters, and the pile yarn demarcates loops fixed in the base weave and is directed in a variety of angles relative to each other.

Patent Document 4 describes a loop part for a hook-and-loop fastener, wherein the loop part includes an interknit base formed from a plurality of interlocked yarn and a plurality of continuous long floats extending separate from the interknit base and joined thereto, such that the long float is formed from monofilament pile yarn, the float is formed in loops for the hook-and-loop fastener, and the loops are arranged in a loop system of the same length or are extending in the same direction.

Patent Document 5 describes a hook-and-loop-type surface fastener female material piece composed of interknitted fabric woven out of a swinging weave of yarn threaded through the rear reed, a loop pile knit weave that engages the swinging weave with yarn threaded through the center reed and is formed with the loop pile next to the weaving direction facing different directions on the left and right, and a base weave that engages the swing weave and loop pile formed weave with yarn threaded through the front reed.

Patent Document 6 describes a surface fastener female material including a fabric composed of warp, weft, and loop yarn wherein the warp is made of a chain braided suture with open laps and close stitches repeating alternatingly, only the close stitch of the warp engages with the open lap of the loop yarn, the loop yarn is fixed to the warp, and the loop yarn protrudes from the front surface on one side of the fabric, alternating left and right relative to the warp.

### PRIOR ART DOCUMENTS

### Patent Documents

Patent Document 1: U.S. Pat. No. 6,096,667 Specification

Patent Document 2: U.S. Unexamined Patent Application No. 2012/0231206 Specification

Patent Document 3: U.S. Pat. No. 6,216,496 Specification

Patent Document 4: U.S. Pat. No. 6,099,932 Specification

Patent Document 5: Japanese Unexamined Patent Application No. 2004-236960

Patent Document 6: International Publication 2010/030548

### SUMMARY OF THE INVENTION

### Problem to be Solved by the Invention

For example, in an absorbent good such as a diaper or the like, when a surface fastener, which is a pair consisting of a female material and a male material, is used as the engagement member, the female material will preferably have a print such as a number, ruled line, or the like applied thereto as an indicator of the engagement position with the male material. For example, if one of two main surfaces is an engagement surface and the other is a base that is a non-engagement surface (for example, a knitted fabric or a non-woven fabric) and a printed layer is formed on the non-engagement surface, a surface fastener female material having a printed layer can be obtained. In this case, the base has an optical transparency such that the printed layer can be recognized through the base. A patterned surface fastener female material can be obtained in this manner. A method wherein a printed film having a print layer support film and a print layer disposed thereon is disposed on the base has been used conventionally as a method for manufacturing a surface fastener female material having a printed layer. However, films usable as a print layer support film include CPP (Cast Polypropylene) film, BOPP (biaxially oriented polypropylene) film and the like, which films are normally not breathable, and bring reduced breathability, flexibility, and stretchability to the good to which the surface fastener female material is attached, compromising the comfort and fit of the absorbent good such as a diaper or the like when wearing it. Thus, it was necessary to have designs for securing breathable parts and stretchable parts by making the surface fastener male material into several strips or the like. Further, the material cost for the print layer support film and the manufacturing cost for the adhesive for adhering the print film to the base cannot be ignored. Thus, there is a demand for a surface fastener female material that has a pattern while avoiding the issues described above.

Knitted fabric used conventionally as a favorable material for obtaining a base having sufficient breathability and engagement properties does not have a non-engagement surface in a form suitable for printing. That is, a favorable print appearance could not be obtained by directly (that is, without a base layer such as a print layer support film and the like) printing on the non-engagement surface. However, sufficient engagement power and durability (particularly durability relating to separation and shear) may not be obtained with a surface fastener female material composed of non-woven fabric when compared with a surface fastener female material made of knitted fabric, despite excellent breathability and printing suitability.

The present invention aims to solve the problems described above, and to provide a surface fastener female material wherein a favorable print appearance can be obtained without using a base layer such as a print layer support film and the like with excellent engagement properties and durability. Further, an embodiment of the present invention aims to provide a surface fastener female material with little lateral difference in engagement power. Further, another embodiment of the present invention aims to provide a surface fastener and absorbent good including these surface fastener female materials.

### Means for Solving the Problem

The present disclosure has at least the following configuration.
(1) A surface fastener female material containing a knitted fabric, wherein

the knitted fabric is woven from a back yarn, a middle yarn, and a front yarn,

the back yarn, the middle yarn, and the front yarn alternatingly form a joining point (I) and a joining point (II),

the joining point (I) is composed of a stitch of the back yarn following an overlap in a first direction or a second direction that is opposite the first direction and an underlap in the first direction in front and back of the overlap, a stitch of the front yarn following an overlap in the same direction as the back yarn and an underlap in the same or opposite direction of the back yarn, and a stitch of the middle yarn following an overlap in the direction opposite the back yarn and the front yarn,

the joining point (II) is composed of a stitch of the back yarn following an overlap in the first or the second direction and an underlap in the second direction in front and back of the overlap, a stitch of the front yarn following an overlap in the same direction as the back yarn and the underlap in the same or opposite direction as the back yarn, and a stitch of the middle yarn following the overlap in the opposite direction of the back yarn and the front yarn,

the front yarn forms a close stitch at the joining point (I), the joining point (II), or at both of these, and

the back yarn contains at least 50% treated yarn by the total thread count standard.
(2) A surface fastener containing the above surface fastener female material and a surface fastener male material.
(³) An absorbent good containing the above surface fastener.

### Effect of the Invention

According to the present invention, a favorable print appearance can be obtained without using a base layer such as a print layer support film, and a surface fastener female material with excellent engagement properties and durability is provided. Further, according to an embodiment of the present invention, a surface fastener female material with little lateral difference in engagement power is provided. According to another embodiment of the present invention, a surface fastener and absorbent good including these surface fastener female materials is provided.

### BRIEF DESCRIPTION OF THE DRAWINGS

FIG. 1 is a schematic diagram illustrating the cross-sectional structure of the surface fastener female material according to an embodiment of the present disclosure.

FIG. 2 is a schematic diagram illustrating the knit weave of the front yarn, middle yarn, and back yarn within the woven fabric containing the surface fastener female material according to an embodiment of the present disclosure.

FIG. 3 is a schematic diagram illustrating the knit weave when the front yarn, middle yarn, and back yarn illustrated in FIG. 2 are each fed one-in-one-out.

FIG. 4 is a schematic diagram illustrating a knitted fabric following the knit weave illustrated in FIG. 2 and FIG. 3.

FIG. 5 is a schematic diagram illustrating the state of the front yarn (left and right, one loop each) formed in the knitted fabric, observed from a cut of the knitted fabric along line A-A (perpendicular to the flow direction of the middle yarn) of FIG. 4.

FIG. 6 is a schematic diagram illustrating the cross-sectional structure of the surface fastener according to an embodiment of the present disclosure.

### BEST MODE FOR CARRYING OUT THE INVENTION

An illustrated configuration of the present invention is described below, but the present invention is not limited to the configurations below, and it is intended that all modifications that do not diverge from the spirit or the range of the scope of the patent are included in the present invention. Further, it is intended that each measurement denoted in the present disclosure is performed by the method described in the [Embodiment] or a method understood to be similar by a person skilled in the art, unless specifically stated.

One aspect of the present disclosure provides

a surface fastener female material containing a knitted fabric, wherein

the knitted fabric is woven from a back yarn, a middle yarn, and a front yarn,

the back yarn, the middle yarn, and the front yarn alternatingly form a joining point (I) and a joining point (II),

the joining point (I) is composed of a stitch of the back yarn following an overlap in a first direction or a second direction that is opposite the first direction and an underlap in the first direction in front and back of the overlap, a stitch of the front yarn following an overlap in the same direction as the back yarn and an underlap in the same or opposite direction of the back yarn, and a stitch of the middle yarn following an overlap in the direction opposite the back yarn and the front yarn,

the joining point (II) is composed of a stitch of the back yarn following an overlap in the first or the second direction and an underlap in the second direction in front and back of the overlap, a stitch of the front yarn following an overlap in the same direction as the back yarn and the underlap in the same or opposite direction as the back yarn, and a stitch of the middle yarn following the overlap in the opposite direction of the back yarn and the front yarn,

the front yarn forms a close stitch at the joining point (I), the joining point (II), or at both of these, and

the back yarn contains at least 50% treated yarn by the total thread count standard.

The surface fastener female material of the present disclosure (also called simply the female member in the present disclosure) may have two main surfaces, one of which may be an engagement surface (that is, a surface intended to engage with the male material) and the other of which may be a non-engagement surface (that is, a surface intended to not engage with the male material). The engagement surface is provided with an engagement element. The engagement element includes a loop engagement element formed by loop-shaped yarn. Thus, the engagement surface of the surface fastener female material can engage the engagement element of the male material (for example, a hook engagement element). The engagement design of a surface fastener in a typical configuration is a hook-and-loop style. A hook-and-loop style surface fastener may be a pair consisting of a male material having hooks composed of protruding objects projecting out in the direction of thickness of the surface fastener, and a female material having loops that can engage the hooks.

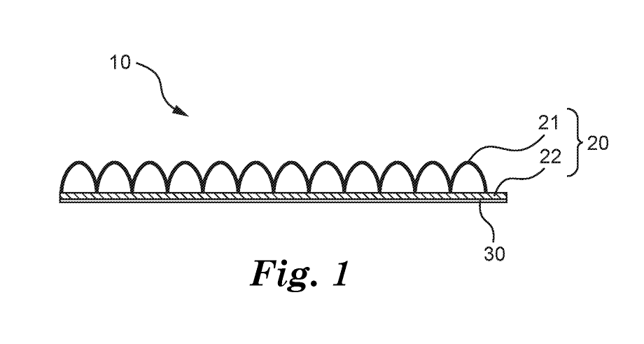

FIG. 1 is a schematic drawing illustrating a cross-sectional structure of the surface fastener female material according to the present disclosure. Referencing FIG. 1, a surface fastener female material 10 of the present disclosure includes a knitted fabric 20. The knitted fabric has a structure such that the yarns are knitted together, and has good breathability, flexibility, and lateral stretchability. These properties are particularly beneficial when the female material is used in absorbent goods such as hygiene goods. Further, the knitted fabric has a structure such that the yarns are knitted together, and may, in some configurations, have good durability compared to a non-woven cloth. The knitted fabric 20 includes a base weave 22 a pile composed of a front yarn 21 on the base weave 22. In a typical configuration, the surface fastener female material 10 can further include a printed part 30 on the knitted cloth 20.

The inventors found the importance of the non-engagement surface being smooth and opaque when applying a print to the non-engagement surface of the surface fastener female material, and further, noticed that the smoothness and opacity could be controlled by controlling the knit weave.

The surface fastener female material of the present disclosure is knitted out of a back yarn, a middle yarn, and a front yarn. This sort of knitted fabric can be knitted by a general knitting machine (for example, a three-bar knitting machine) provided with a back bar, middle bar, and front bar. Note that, for example, in configurations using elastic yarn as described hereafter, a four-bar knitting machine or the like may be used. The knitted fabric according to the present disclosure can be produced quickly and at low cost using a general knitting machine. In a typical configuration, the back bar threads the back yarn, the middle bar threads the middle yarn, and the front bar threads the front yarn. As for the threading design, there is through threading, one-in-one-out (that is, pulling out yarn skipping every other yarn), one-in-two-out (that is, threading one and pulling out two) and the like. One-in-one-out is beneficial considering the points of breathability, flexibility, and stretchability, as well as for forming the structure of the female material of the present disclosure.

The back yarn and middle yarn together compose the base weave 22 of the knitted fabric 20, and contributes to the attachment of the female material to the subject of application (for example the fabric of an absorbent material) of the subject good (for example, the absorbent good). The front yarn 21 is mainly exposed on the first front surface of the knitted fabric and forms a loop engagement element. Thus, the first front surface of the knitted fabric can function as an engagement surface (that is, a surface to be engaged with the male material) on the female material. The back yarn is mainly exposed on the second front surface opposing the first front surface of the knitted fabric. The second front surface is the non-engagement surface of the female material. The non-engagement surface can function as the attachment surface when attaching the female material to the subject good.

In a typical configuration of the present disclosure, the female material further has a print part. As illustrated in FIG. 1, the print part 30 is directly disposed on the non-engagement surface of the base weave 22 of the knitted fabric 20. That is, the print part 30 may be provided on the non-engagement surface of the base weave 22 of the knitted fabric 20. Further, one part of the print part 30 may exist between each yarn (back yarn, middle yarn) or the like composing the base weave 22 of the knitted fabric 20. In the present disclosure, it is intended that the non-engagement surface is smooth and opaque for favorable application of a print (especially a coating-based print). The female material of the present disclosure has, in addition to having good breathability, flexibility, and stretchability because of the knitted fabric, the benefit of the print part being able to be disposed on the knitted fabric without a base layer (for example, a print layer support film or the like). That is, while the base layer may compromise the breathability, flexibility, and stretchability of the female material, in the female material of the present disclosure, there is no such base layer, and the print part can be directly disposed on the knitted fabric. This allows for a female material having a printed part that also has breathability, flexibility, and stretchability. Note that in the present disclosure, the smoothness of the non-engagement surface is affected by the difference between the thickness of the knitted cloth in the weaving part (joining part) and the thickness of the knitted fabric between weaving parts (the region between two neighboring middle yarns). The smaller this difference is, the smoother it is. Also, the opacity can be judged by the optical transmittance of the fabric, Lab color space (L*) and an evaluation of the appearance of the non-engagement surface. The opacity is higher with lower optical transmittance and a higher Lab color space (L*) value. The appearance of the non-engagement surface can be checked with a visual observation of the appearance or the like.

Note that the non-engagement surface having a favorable smoothness and opacity further has the following benefits when the surface fastener female material of the present disclosure is used in absorbent goods uses (for example, a diaper). In an assembly line for an absorbent good, conveyance via a suction hole provided on a suction roll is necessary for attaching the surface fastener to the body of the absorbent good. At this time, if there are too many gaps in the surface fastener, or the bumps on the surface of the surface fastener facing the suction roll are too large, conveyance of the surface fastener female material via suction roll becomes difficult, which may raise issues such as reduced productivity. The surface fastener female material of the present disclosure avoids these issues, and has the benefit of contributing to good productivity on a production line for an absorbent good (for example, a diaper).

In the knitted fabric, the back yarn, middle yarn, and front yarn alternatingly form joining point (I) and joining point (II). Joining point (I) is composed of a stitch of the back yarn following an overlap in a first direction or a second direction that is opposite the first direction and the underlap in the first direction in front and back of the overlap, a stitch of the front yarn following an overlap in the same direction as the back yarn and the underlap in the same or opposite direction of the back yarn, and a stitch of the middle yarn following an overlap in the direction opposite the back yarn and the front yarn; joining point (II) is composed of a stitch of the back yarn following an overlap in the first or the second direction and the underlap in the second direction in front and back of the overlap, a stitch of the front yarn following an overlap in the same direction as the back yarn and the underlap in the same or opposite direction as the back yarn, and a stitch of the middle yarn following the overlap in the opposite direction of the back yarn and the front yarn. Further, the front yarn forms a close stitch at joining point (I), joining point (II), or at both of these. By having this knit weave, the front yarn, in essence, has a structure such that it protrudes alternating left and right from the middle yarn on the surface of one side of the knitted fabric.

In a knit weave as described above, the weave can have good engagement strength and durability because the front yarn and back yarn are firmly fixed at joining point (I) and (II). When manufacturing the surface fastener female material (knitted fabric), and further, when some time has passed after manufacturing, it is difficult for backside protrusion of the front yarn protruding from the base weave of the surface fastener female material to occur (that is, a state wherein the pile of front yarn formed on one surface of the knitted fabric composing the surface fastener female material is formed on the opposite surface of the knitted fabric). Thus, the obtained surface fastener female material has excellent engagement power with the male material as well as durability (for example, tearing strength).

Here, “the front yarn protrudes alternating left and right from the middle yarn on the surface of only one side of the knitted fabric” means that in the knitted fabric 20, when looking at the cross section in the direction perpendicular to the middle yarn direction, the front yarn 21 is formed alternating left and right side keeping an angle with the source at the middle yarn joining part relative to the base weave 22. This front yarn can easily engage the engagement element of the male material, and contributes to excellent engagement properties.

In the illustrated configuration, the back yarn is knitted having a weave pattern of a stitch forming the joining point (I)/blind lap/stitch forming joining point (II)/blind lap, and the front yarn is knitted having a weave pattern of stitch forming joining point (I)/open lap/stitch forming joining point (II)/open lap, and the middle yarn is knitted having a chain weave pattern.

In the surface fastener female material, it is preferable for the lateral difference in engagement power to be small (that is, the lateral difference in power felt when separating the female material from the male material). When the joining point (I) and (II) are aligned to the same wale and the front yarn is reeled out the same swing width on the left and right centered on the joining point (I) and (II), it contributes to an engagement power with little lateral difference, which is preferable. Further, when the joining point (I) and (II) are aligned to the same wale and the back yarn is reeled out the same swing width on the left and right centered on the joining point (I) and (II), it contributes to better opacity and smoothness, which is preferable.

In the illustrated configuration, as examples of each knit weave of the front yarn, middle yarn, and back yarn, an object as shown in Table 1 can be illustrated. The joining point (I) and (II) can be composed by combining the knit weave, for example, according to number 1 through 24 of Table 1.

TABLE 1

| Number | Front Yarn | Middle Yarn | Back Yarn |
|---|---|---|---|
| 1 | (A)-(A + 1)/xx/(A + 1)-(A)/yy | 1-0/0-1/0-1/1-0 | (C)-(C + 1)/0-0/(C + 1)-(C)/D-D |
| 2 | (A)-(A + 1)/xx/(A + 1)-(A)/yy | 1-0/0-1/0-1/1-0 | (C)-(C + 1)/D-D/(C + 1)-(C)/0-0 |
| 3 | (A)-(A + 1)/xx/(A + 1)-(A)/yy | 1-0/1-0/0-1/0-1 | (C)-(C + 1)/0-0/(C + 1)-(C)/D-D |
| 4 | (A)-(A + 1)/xx/(A + 1)-(A)/yy | 1-0/1-0/0-1/0-1 | (C)-(C + 1)/D-D/(C + 1)-(C)/0-0 |
| 5 | (A)-(A + 1)/xx/(A + 1)-(A)/yy | 1-0/0-1/0-1/0-1 | (C)-(C + 1)/0-0/(C − 1)-(C)/D-D |
| 6 | (A)-(A + 1)/xx/(A + 1)-(A)/yy | 1-0/1-0/0-1/1-0 | (C)-(C + 1)/0-0/(C + 1)-(C)/D-D |
| 7 | (A)-(A + 1)/xx/(A + 1)-(A)/yy | 1-0/1-0/0-1/1-0 | (C)-(C + 1)/D-D/(C + 1)-(C)/0-0 |
| 8 | (A)-(A − 1)/xx/(A − 1)-(A)/yy | 1-0/0-1/0-1/0-1 | (C)-(C + 1)/D-D/(C + 1)-(C)/0-0 |
| 9 | (A + 1)-(A)/xx/(A + 1)-(A)/yy | 0-1/1-0 | (C + 1)-(C)/0-0/(C + 1)-(C)/D-D |
| 10 | (A + 1)-(A)/xx/(A + 1)-(A)/yy | 0-1/1-0 | (C + 1)-(C)/D-D/(C + 1)-(C)/0-0 |
| 11 | (A + 1)-(A)/xx/(A + 1)-(A)/yy | 0-1/0-1 | (C + 1)-(C)/0-0/(C + 1)-(C)/D-D |
| 12 | (A + 1)-(A)/xx/(A + 1)-(A)/yy | 0-1/0-1 | (C + 1)-(C)/D-D/(C + 1)-(C)/0-0 |
| 13 | (A + 1)-(A)/xx/(A − I)-(A)/yy | 0-1/1-0/0-1/0-1 | (C + 1)-(C)/0-0/(C + 1)-(C)/D-D |
| 14 | (A + 1)-(A)/xx/(A + 1)-(A)/yy | 0-1/1-0/0-1/0-1 | (C + 1)-(C)/0-0/(C + 1)-(C)/0-0 |
| 15 | (A + 1)-(A)/xx/(A + 1)-(A)/yy | 0-1/0-1/0-1/1-0 | (C + 1)-(C)/0-0/(C + 1)-(C)/D-D |
| 16 | (A + 1)-(A)/xx/(A + 1)-(A)/yy | 0-1/0-1/0-1/1-0 | (C + 1)-(C)/D-D/(C + 1)-(C)/0-0 |
| 17 | (A)-(A + 1)/xx/(A)-(A + 1)/yy | 1-0/0-1 | (C)-(C + 1)/D-D/(C)-(C + 1)/0-0 |
| 18 | (A)-(A + 1)/xx/(A)-(A + 1)/yy | 1-0/0-1 | (C)-(C + 1)/0-0/(C)-(C + 1)/D-D |
| 19 | (A)-(A + 1)/xx/(A)-(A + I)/yy | 1-0/1-0 | (C)-(C + 1)/D-D/(C)-(C + 1)/0-0 |
| 20 | (A)-(A + 1)/xx/(A)-(A + 1)/yy | 1-0/1-0 | (C)-(C + 1)/0-0/(C)-(C + 1)/D-D |
| 21 | (A)-(A + 1)/xx/(A)-(A + 1)/yy | 1-0/0-1/1-0/1-0 | (O-(C + 1)/D-D/(C)-(C + 1)/0-0 |
| 22 | (A)-(A + 1)/xx/(A)-(A + 1)/yy | 1-0/0-1/1-0/1-0 | (C)-(C + 1)/0-0/(C)-(C + 1)/D-D |
| 23 | (A)-(A + 1)/xx/(A)-(A + 1)/yy | 1-0/1-0/1-0/0-1 | (C)-(C + 1)/0-0/(C)-(C + 1)/D-D |
| 24 | (A)-(A + 1)/xx/(A)-(A + 1)/yy | 1-0/1-0/1-0/0-1 | (C)-(C + 1)/D-D/(C)-(C + 1)/0-0 |

In Table 1, xx is 0-1 or 1-0, yy represents (B)−(B+1) or (B+1)−(B), A and C are whole numbers greater than or equal to 1, B is a whole number greater than or equal to 2, D is a whole number greater than or equal to 3, where A<B, and C+1<D. In a preferable configuration, xx is 0-1. In a preferable configuration, yy is (B+1)−(B).

From the standpoint of having the front yarn reeled out with the same swing width on the left and right centered on the joining point (I) and (II), it is preferable for the relationship B=A×2 to be true. Further, from the standpoint of having the back yarn reeled out with an even swing width on the left and right centered on the joining point (I) and (II), it is preferable for the relationship D=C×2+1 to be true.

Above all, from the standpoint of engagement power, the knit weave is preferably number 1 through 8 or number 17 through 24, and more preferably number 1 through 8.

An example with a knit pattern such that the front yarn is 1-2/0-1/2-1/3-2, the middle yarn is 1-0/0-1/0-1/1-0, and the back yarn is 2-3/0-0/3-2/5-5, that is, a knit pattern such that within number 1 of Table 1, A is 1, xx is 0-1, yy is (B+1)−(B), B is 2, C is 2, and D is 5 is described below as an example.

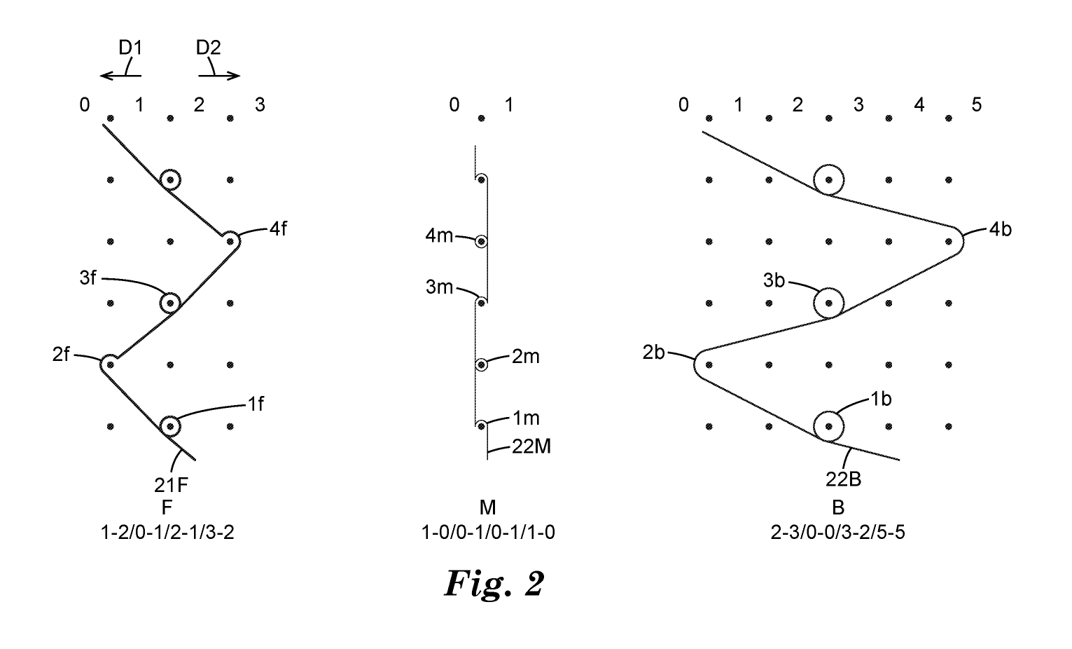

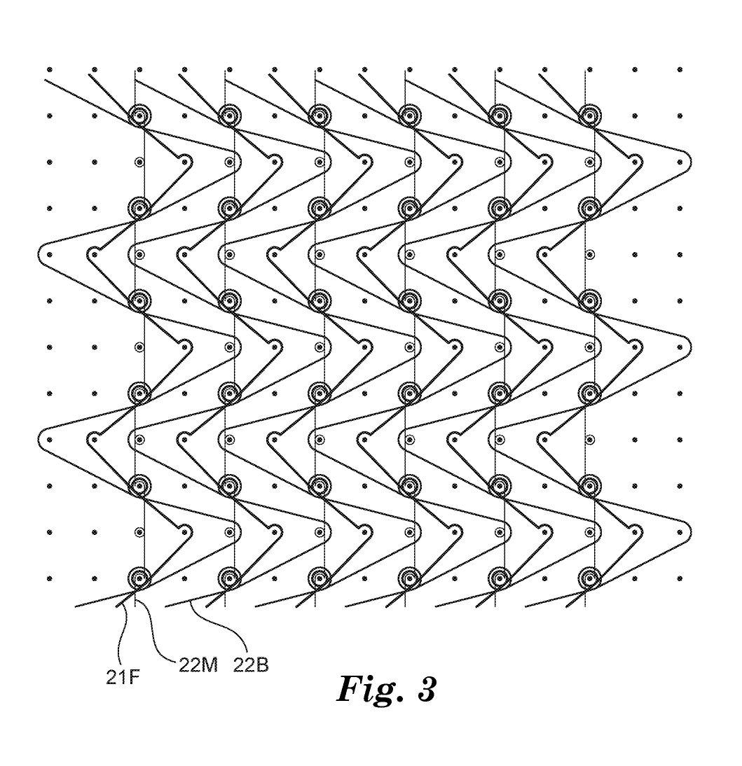

FIG. 2 is a schematic diagram illustrating a knit weave of the front yarn, middle yarn, and back yarn within a woven fabric containing the surface fastener female material according to an embodiment of the present disclosure, and FIG. 3 is a schematic diagram illustrating the knit weave when the front yarn, middle yarn, and back yarn illustrated in FIG. 2 are each fed one-in-one-out.

Referencing FIG. 2 and FIG. 3, the pattern of each yarn is described below, with the direction of arrow D1 as the first direction and the direction of arrow D2 as the second direction. A front yarn 21F has a knit pattern of a stitch if following an overlap in the second direction and an underlap in the first direction in front and back of the overlap, an open lap 2f, a stitch 3f following an overlap in the first direction and an underlap in the second direction in front and back of the overlap, and an open lap 4f. The back yarn 22B has a knit pattern of a stitch 1b following an overlap in the second direction and an underlap in the first direction in front and back of the overlap, an insert weave part 2b made by blind lapping (that is, a yarn movement in a knit weave represented by N-N (N is an integer greater than or equal to 0), wherein the yarn is not wound around the needle (thus, not forming a stitch) and sent out), a stitch 3b following an overlap in the first direction and an underlap in the second direction in front and back of the overlap, and an insert weave part 4b made by blind lapping. The middle yarn 22M has a chain knit pattern of a stitch 1m following an overlap in the first direction, a stitch 2m following an overlap in the second direction, a stitch 3m following an overlap in the second direction, and a stitch 4m following an overlap in the first direction. Stitches 1f, 1b, and 1m form joining point (I), and stitches 3f, 3b, and 3m form joining point (II). Thus, joining point (I) and joining point (II) are formed alternatingly. The back yarn 22B and the middle yarn 22M form the base weave, and the front yarn 21F becomes the pile of the knitted fabric, being reeled out on the left and right of the middle yarn 22M.

At joining point (I), the stitch 1f and the stitch 1b are following the same overlap direction, and the stitch 1m follows the overlap direction opposite to these. Further, at joining point (II), the stitch 3f and the stitch 3b are following the same overlap direction, and the stitch 3m follows the overlap direction opposite to these. In FIG. 2 is shown an example where, as stitch 1f, 3f, 1b, and 3b, the overlap direction is opposite the direction of the underlap in front and back of the overlap (that is, a close stitch). However, as can be seen in Table 1, in the present disclosure, the overlap and the underlap may be in the same direction as each other (that is, an open lap). However, the front yarn forms a close stitch at joining point (I), joining point (II), or both. Further, in FIG. 2 is shown an example where within joining point (I) and (II), the underlap direction is the same at stitch 1f and stitch 1b, and is the same at stitch 3f and 3b. However, as seen in Table 1, within the present disclosure, the underlap directions may be opposite to each other.

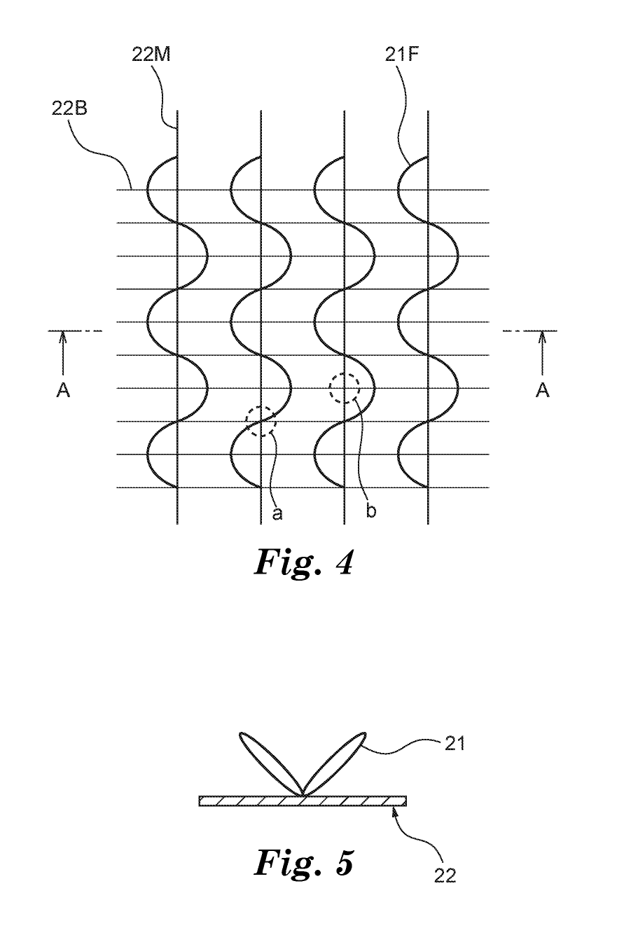

FIG. 4 is a schematic diagram illustrating a knitted fabric following the knit weave illustrated in FIG. 2 and FIG. 3. FIG. 5 is a schematic diagram illustrating the state of the front yarn (left and right, one loop each) formed in the knitted fabric, observed from a cut of the knitted fabric along line A-A (perpendicular to the flow direction of the middle yarn) of FIG. 4. Referencing FIG. 4, in the knit weave shown in FIGS. 2 and 3, the middle yarn 22M alternatingly forms joining point a and intersecting point b of the middle yarn and the back yarn. Further, the front yarn 21F protrudes alternating left and right of the middle yarn 22M from the surface of one side of the knitted fabric. Referencing FIG. 5, the protrusion angle of the front yarn 21 relative to the base weave 22 of the knitted fabric is, from the standpoint of engagement power, preferably greater than or equal to 30 degrees, and in some configurations, may be greater than or equal to 45 degrees. Further, there is not particularly an upper limit to the protrusion angle, and the protrusion angle of the front yarn 21 relative to the base weave 22 may be 80 degrees, or further, 90 degrees.

The pattern of the knit weave of the back yarn, middle yarn, and front yarn may be one type over the entire knitted fabric, or a plurality of types may be mixed. A person skilled in the art may appropriately set a knitted weave from one type or a plurality of types of patterns throughout the knitted fabric according to the threading method of the knitting machine and the controls of the knitting program.

The back yarn, middle yarn, and front yarn may each be one filament or a bundle of a plurality of filaments (that is, multi-filament). In a preferable configuration, the middle yarn is monofilament, from the standpoint of smoothness. It may be preferable to use a multi-filament over a monofilament for the front yarn, from the standpoint of increasing the engagement likelihood with the male material. In this case, the filaments of the front yarn may break when engaging with the male material if they are too thin, so based on the shape and such of the surface fastener, a suitable thickness is selected.

Generally, the total fineness of the filaments of the back yarn, middle yarn, and front yarn may be from about 20 to about 220 dtex, or from about 20 to about 100 dtex. The fineness of a monofilament may be from about 0.5 to about 7 dtex.

The material quality of the back yarn, middle yarn, and front yarn may each be one type or two or more types, and can be designed according to the desired properties of the knitted fabric. Further, the varieties of the fibers composing each of the back yarn, middle yarn, and front yarn may be one type or two or more types.

As for the material quality of the middle yarn and front yarn, each of them may be, for example, polyolefin (for example, polyethylene, polypropylene, and the like), polyester, polyamide, polyurethane, EVA (ethylene vinyl acetate), polylactate, rayon, a copolymer or mixture of these, or a natural fiber or the like. In some configurations, from the standpoint of preventing destruction of the female material due to engagement with the male material, a high-strength polyamide is used. Further, if considering material cost and environmental stability, polyester is preferred.

As for the material quality of the back yarn, it may be, for example, polyolefin (for example, polyethylene, polypropylene, and the like), polyester, polyamide, polyurethane, EVA (ethylene vinyl acetate), polylactate, or a copolymer or mixture of these. In some configurations, from the standpoint of preventing destruction of the female material due to engagement with the male material, a high-strength polyamide is used. Further, if considering material cost and environmental stability, polyester is preferred.

In the present disclosure, the back yarn contains at least about 50% treated yarn by the total thread count standard. Further, in a preferred configuration of the present disclosure, in addition to the back yarn, the front yarn also contains at least about 50% treated yarn of the total thread count standard. In the present disclosure, at least about 50% by the total thread count standard of the back yarn and the front yarn refers to at least about 50% of the total thread count of the back yarn (that is, the thread count of back yarn arranged in the wale direction on the knitted fabric) and at least about 50% of the total thread count of the front yarn (that is, the thread count of front yarn arranged in the wale direction on the knitted fabric).

In the present disclosure, treated yarn includes all yarn which has been given a treatment for providing bulkiness and/or elasticity (for example, crimpability). A typical configuration may be a yarn provided with bulkiness, crimpability, and the like by heat-setting (applying dry heat or wet heat) while twisted. Treated yarn may be, for example, manufactured via the twist-untwist method, the false-twist method, or the like. In particular, false twist treated yarn, also called textured yarn (for example, 1-heater false twist treated yarn) is beneficial from the standpoint of having better smoothness and opacity of the non-engagement surface, because it has the tendency to expand favorably so as to fill in the gaps between stitches in the knitted fabric. Note that concerning the twist count of the treated yarn, it is preferable for the lower limit to be at least about 2000 tpm (tpm: twist per meter) or at least about 2500 tpm, and the upper limit to be at most about 6000 tpm or at most 5000 tpm, from the standpoint of non-engagement surface opacity in the surface fastener female material obtained.

As for treated yarn, both S-twist yarn (that is, yarn twisted in the S-direction), and Z-twist yarn (that is, yarn twisted in the Z-direction) may be used. From the standpoint of preventing curling of the knitted fabric, it is preferable to use a combination of S-twist yarn and Z-twist yarn. The benefit of preventing curling is that it is sensible in the manufacturing process of an absorbent good using the surface fastener female material. Specifically, curl prevention contributes to higher efficiency when conveying the surface fastener female material via suction roll as described previously. In a preferable configuration, the back yarn and/or the front yarn has disposed alternatingly one or a plurality of S-twist yarn and one or a plurality of Z-twist yarn in the direction of the wale. For example, the S-twist yarn and Z-twist yarn may be disposed alternating every yarn in the wale direction like SZSZSZ, and the S-twist yarn and Z-twist yarn may be disposed alternating every few yarns in the wale direction like SSZZSSZZSSZZ or the like. From the standpoint of curl prevention, in a preferable configuration as treated yarn, the S-twist yarn and the Z-twist yarn is disposed alternating every yarn (that is, like SZSZSZ) in the wale direction.

The treated yarn may have elasticity. Elasticity may be provided by the elasticity of the material quality composing the yarn, by the twisting method of the yarn, or a combination of these.

In some configurations, the back yarn and/or the front yarn further include an elastic yarn having the same or smaller fineness than the treated yarn. It is intended that the elastic yarn is not a treated yarn. Polyurethane elastic yarn is an example of an elastic yarn. In the illustrated configuration, the back yarn and/or the front yarn has one or a plurality of treated yarn and one or a plurality of elastic yarn disposed alternatingly in the wale direction. For example, several treated yarns and one elastic yarn may be disposed alternatingly. In a preferable configuration, the fineness (total fineness) of the elastic yarn is at least ⅓ and no more than 1 of the fineness of the treated yarn. For example, if the treated yarn is 84 dtex, the fineness of the elastic yarn may be at least about 28 dtex, and no more than about 84 dtex.

Treatment for treated yarn is generally unwound relatively easily, and individual threads are easily dispersed. It is beneficial for at least about 50% of the back yarn to be treated yarn from the standpoint of good smoothness and opacity. In a preferable configuration, the lower limit of treated yarn in the back yarn is at least about 55%, at least about 60%, or at least about 67%, while the upper limit is at most about 100%, at most about 90%, or at most about 80%. Further, if the back yarn further includes an elastic yarn or the like in addition to treated yarn as described previously, from the standpoint of obtaining a favorable benefit therefrom, the treated yarn may be from about 50% to about 80% if the total of treated yarn and elastic yarn is 100%. Note that when inserting elastic yarn into the back yarn, the elastic yarn may be put in with a different beam than the treated yarn. That is, in addition to the method of winding the elastic yarn and treated yarn in the same beam, the elastic yarn may be inserted into the back yarn with a different beam than the treated yarn.

Further, it is beneficial for at least about 50% of the front yarn to be treated yarn, from the standpoint of having good engagement power and durability. In a preferable configuration, the lower limit of treated yarn in the front yarn is at least about 55%, at least about 60%, or at least about 67%, while the upper limit is at most about 100%, at most about 90%, or at most about 80%. Further, if the front yarn further includes an elastic yarn or the like in addition to treated yarn as described previously, from the standpoint of obtaining a favorable benefit therefrom, the treated yarn may be from about 50% to about 80% if the total of treated yarn and elastic yarn is 100%. Note that when inserting elastic yarn into the front yarn, the elastic yarn may be put in with a different beam than the treated yarn. That is, in addition to the method of winding the elastic yarn and treated yarn in the same beam, the elastic yarn may be inserted into the front yarn with a different beam than the treated yarn.

The middle yarn may be treated yarn or untreated yarn (that is, yarn that is not treated).

The female member of the present disclosure may arbitrarily have, in addition to the knit fabric, a print part and further additional elements. In a preferable configuration, the print part is formed by a coating on the knit fabric. The non-engagement surface of the knit fabric according to the present disclosure has excellent smoothness and opacity, allowing a print part with a good outer appearance to be formed even when the print part is disposed directly on the knit fabric. In particular, the good opacity of the knit fabric, and particularly that of the non-engagement surface, is beneficial when forming a print part via a coating.

Additional elements may be imparting adhesiveness via an adhesive treatment, imparting strength with a resin treatment, a knitted fabric, a woven cloth, a non-woven cloth, paper, or a laminate of these, or the like. The lamination method is not particularly limited, and conventional known methods such as coating, dry laminate, extrusion laminate, wet laminate, heat laminate, ultrasonic, and the like may be used.

The print part 30 can be directly fixed onto the knitted fabric 20. The printed part being directly fixed onto the knitted fabric means the printed part is disposed on the knitted fabric without another member or layer (in other words, is touching the knitted fabric) and the print part essentially cannot be removed from the knitted fabric while keeping its shape. That is, as described above, the print part 30 can be provided on the non-engagement surface of the base weave 22 of the knitted fabric 20. Further, one part of the print part 30 may exist between each yarn (back yarn, middle yarn) composing the base weave 22 of the knitted fabric 20, or the like. In this manner, the shape of the print part is maintained by the knitted fabric, and the print part is supported on the knitted fabric with the knitted fabric as the supporting body. Typically, the print part in a female material is not fixed to a sublayer or other member such as a print layer support film or the like, but exists in a state of being fixed only to the knitted fabric. Thus, for example, a female material made of a print sheet having a print layer support film and an ink layer provided thereon that is stacked on a knitted fabric is distinguished from a female material with the print part directly fixed on the knitted fabric, as the print layer along with the print layer support film can usually be separated from the base layer.

The print part may be composed of only ink, or in addition to ink, may have a base coat and/or a top coat. Ink and a given base coat and top coat may each exist as a continuous layer on the knitted fabric, or may be disposed discontinuously, and designed according to the desired purpose, for example a purpose of an ink design. The design may be selected at will, and may be a character, picture, shape, or the like.

The ink material may be any conventionally known ink, and water-based and solvent-based inks are used. Conventional resins used normally can be used as a resin contained in the ink. For example, acrylic resin, polyurethane resin, polyamide resin, urea resin, polyester resin, vinyl chloride resin, epoxy resin, polyvinyl alcohol, ethylene-vinyl acetate copolymer resin, olefin resin, epoxy resin, petroleum-based resin, cellulose derivative resin, and natural resins such as rosin derivative resin and the like can be used. By using an ink with excellent fastness, even when ink is exposed during use of a good having the female material of the present disclosure, the ink will not come off easily. From this standpoint, for example, urethane-based ink, epoxy-based ink, and the like are preferable. Further, the ink itself may be a hot-melt type adhesive.

In a typical configuration, the print part may be formed via a coating on the non-engagement surface of the knitted fabric. A conventionally known method used for printing via a coating can be used for the coating. Having the print part formed via coating is beneficial for the point that the reduction in breathability and flexibility occurring in female materials having, for example, a print layer support film, can be avoided. In some configurations, a female member of the present disclosure further having a print part via coating may further have good breathability, flexibility, and stretchability. These benefits are beneficial for comfort when, for example, the female member is used in an absorbent good such as a hygiene good.

The female member of the present disclosure is beneficial from a material cost and manufacturing cost perspective because the use of materials that bring a cost increase (for example, a print layer support film, adhesive, and the like) can be eliminated, making it possible to manufacture with a simple configuration and manufacturing method.

In a preferred embodiment, the female member may have a basis weight of about 10 to about 100 g per 1 square meter. If the basis weight is greater than or equal to about 10 g/m², the rigidity of the female material does not become too large, and the flexibility of the female material is good.

The surface fastener female material 10 of the present invention has excellent engagement power with a male material, and further, in certain configurations, has little lateral difference in engagement power, allowing for a smaller basis weight compared to the conventional item. As such, a surface fastener female material with excellent visibility can be manufactured at low cost.

In a preferable configuration, the female material is supple and flexible. For example, the rigidity of the female material is preferably at least about 0.8 N/inch and at most about 3.5 N/inch in the MD direction (middle yarn direction), and at least about 0.3 N/inch and at most about 3.5 N/inch in the CD direction (back yarn direction) that makes a 90° angle with the MD direction. When the rigidity is within the above range, the female material has good machine strength while having excellent flexibility.

In a favorable configuration, the female material has excellent breathability. More specifically, the breathability of the female material as measured by the Gurley method is preferably about 5 seconds/100 cc or less, from the standpoint of providing good comfort to a hygiene product. More preferable is about 3 seconds/100 cc or less, and further preferable is about 1 second/100 cc or less. There is no particular limit for the lower limit, but in a given configuration, is about 0.1 seconds/100 cc or more.

Additional treatments such as raising, emboss, dyeing, or coloring may be further applied to the surface fastener female material 10. Further, the knitted fabric 20 may be entirely or partially dyed. Particularly when used as a member in an absorbent good such as a diaper, gloss known as sheen or shine can be eliminated, allowing an aesthetically pleasing appearance to be obtained.

Another configuration of the present disclosure provides a surface fastener including a surface fastener female material described above and a surface fastener male material (also called simply male material in the present disclosure).

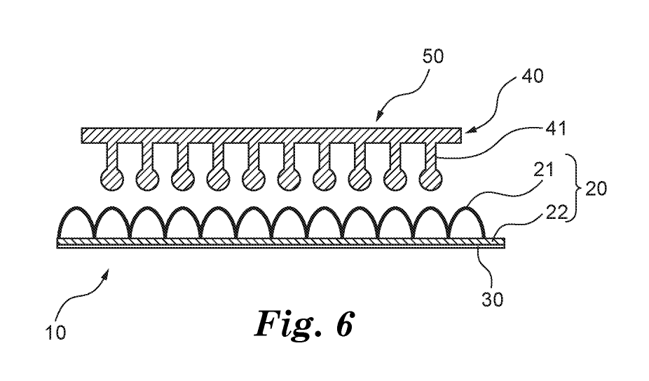

FIG. 6 is a schematic diagram illustrating the cross-sectional structure of the surface fastener according to an embodiment of the present disclosure. Referencing FIG. 6, a male material 50 usually has a sheet-shaped base part 40 and a plurality of hook engagement elements 41 disposed as protruding objects on the base. The shape of the hook engagement elements is not particularly limited, but may be pillar-shaped bodies such as a cylindrical body, a rectangular prism, or a hexagonal pillar, and for improving mechanical engagement operation, may have a disc-type, mushroom-type, key-type, wedge-type, arrowhead-type, or the like as a top end part. The top end part can mechanically engage well with the corresponding engagement surface, and can easily be attached and removed.

In a surface fastener having a surface fastener female material 10 of the present invention, the front yarn 21 protrudes alternating left and right from one surface of the knitted fabric 20 relative to the middle yarn, and the female material has a structure such that the front yarn cannot easily protrude from the back side; when engaging the male material, the lateral difference in engagement power with the male material is small.

Here, the lateral difference in engagement power can be evaluated by engaging the surface fastener female material with the male material, and in the width direction (that is, the direction perpendicular to the C direction, D direction and middle yarn direction), measuring the engagement power when separating them from the right to the left (right engagement power) and the engagement power when separating them from the left to the right (left engagement power) at a plurality of points, and finding the absolute value of the value represented by {(average value of the measured right engagement power)—(average value of the measured left engagement power)}/(average value of all measured values for right engagement power and left engagement power).

The lateral difference in engagement power is smaller as the obtained value approaches zero.

For example, the concentration of hook engagement elements in the male material is generally from approximately 500 to 5000 per square inch, and in a configuration, a concentration of 1600 per square inch is used.

The material for the engagement element of the male material may be a variety of thermoplastic resin materials, such as, for example, polyethylene (for example, high-density polyethylene), polypropylene, polystyrene, polyvinyl chloride, polyethylene terephthalate, polybutylene terephthalate, nylon, polycarbonate, polymethyl methacrylate, polyacetal, polymethyl pentene, acrylonitrile-styrene-butadiene, polyphenylene ether, polyphenylene sulfide, as well as a styrene-based elastomer such as styrene-butadiene-styrene, styrene-isoprene-styrene and the like, an olefin-based elastomer such as ethylene-α-olefin copolymer and the like, an ester-based elastomer, amide-based elastomer, urethane-based elastomer, vinyl chloride-based elastomer, silicone-based elastomer, fluoride-based elastomer, and alloys thereof, and the like. Hook tape (CS-600) marketed by 3M Japan (Corp.) and the like are examples of a male material.

The surface fastener of the present disclosure can be used in a variety of goods, such as, for example, absorbent goods including paper diapers, sanitary napkins, breast milk pads, and the like; fixing tools for floors and walls; fixing tools for clothing; fixing tools for cleaning supplies; fixing tools for automotive interiors, and the like. The surface fastener of the present disclosure is particularly preferable as a surface fastener for absorbent goods (for example, hygiene goods such as paper diapers and the like) because it can have good breathability, flexibility, and stretchability because of its composition.

In some configurations, when the surface fastener is used in an absorbent good, or especially a paper diaper, it is preferable to consider the engagement properties of the female material and the male material, and especially the performance of the female material. In a standard paper diaper, male material is provided as a pair on the left and right side of the back of the wearer, and a female material is provided as a pair on the left and right side of the front side of the body near the stomach. Thus, the male member and female member in the two places on the left and right of the diaper will each be engaged, and a small lateral difference in engagement power, that is, the difference in power for separating in the left direction and the power for separating in the right direction will be preferable from the standpoint of a secure fix for the paper diaper by the surface fastener.

Absorbent goods include diapers for kids, adults, and pets, and napkins for hygiene and other uses. In a typical configuration, an absorbent good is a diaper. The good smoothness and opacity of the knitted fabric according to the present disclosure is beneficial in stable conveying of the diaper by a suction hole provided on a suction roll in an assembly line.

In a preferable configuration, an absorbent good has excellent breathability. More specifically, the breathability of the absorbent good as measured by the Gurley method is preferably about 5 seconds/100 cc or less, from the standpoint of providing good comfort to a hygiene product. More preferable is about 3 seconds/100 cc or less, and further preferable is about 1 second/100 cc or less. There is no particular limit for the lower limit, but in a given configuration, is about 0.1 seconds/100 cc or more.

The manufacturing method for the absorbent good is not particularly limited, and may be, for example, one of the following methods. Conventionally known elements may be used for elements of the absorbent good other than the surface fastener, and will not be described in detail here. In the absorbent good, the method for attaching the surface fastener to the subject of application may be any method that is conventionally known. One surface of the surface fastener (for example, on the female material, the surface with a print part formed thereon) can be joined to the subject of application using a conventionally known joining method (adhesion via glue, heat-fusing, supersonic treatment, and the like, sewing, mechanical fixing via a stapler or the like). For fixing with glue, a known adhesive is selected according to need, such as synthetic rubber-based including SIS, SBS, and the like, olefin-based, acrylic-based, silicone-based, EVA, and the like, and is not limited to these resins.

### [Embodiment]

An illustrated configuration of the present invention is described further below with an embodiment, but the present invention is not limited to such.

### (Method for Evaluation)

1. 135° Separation Test

A test piece of MD direction length 25 mm×CD direction length 20 mm was cut out of a male material having hooks (3M mechanical fastener hook NC-2050, commercially available from 3M Japan (Corp.)) without damaging the hooks. The obtained test piece was attached to the pressure-sensitive adhesion surface of a standard fixing tape (4591LH (commercially available from 3M Japan (Corp.)). The remaining portion of the pressure-sensitive adhesion surface (that is, the exposed portion of pressure-sensitive adhesion surface) was covered with paper. A female material test piece of MD direction length 50 mm×CD direction length 100 mm was attached to a test panel with double-sided tape (Y9448 (commercially available from 3M Japan (Corp.)). Matching the CD direction of the male material test piece with the CD direction of the female material test piece, the male material test piece was gently placed on the female material test piece, and a 2.0 kg hand roller was ran over it one time back and forth in the CD direction at about 300 mm/minute.

The test panel was placed in a 135° separation jig. The paper end portion of the male material test piece was attached to the upper chuck of the tensile testing machine. The starting point for separation was made to be directly below the upper chuck. In the separation test, measurements were taken with the test panel being pushed down without it moving. The average of the separation chart (area average) was recorded as the engagement power.
2. MD Tensile Strength

A test piece of MD direction 50 mm and CD direction about 25 mm was cut out of the knitted fabric and secured in the chuck of the Tensilon Tensile tester ((Corp). OrionTech, RTC-1225), pulled longitudinally at a speed of 300 mm/minute, the stretch distance during the stretch process and the corresponding tensile load was plotted, and a relationship curve of the stretch distance and the tensile load was obtained (horizontal axis: stretch distance (mm), vertical axis: tensile load (N)). The tensile load value when the stretch ratio was 5% was taken from the obtained relationship curve.
3. Optical Transmittance

The optical transmittance of the knitted fabric was measured in compliance with the total light transmittance test method (JIS K7361). The NDH2000 made by Nippon Denshoku Kogyo (Corp.) was used as the measurement device. A 50 mm×50 mm test piece cut out of the knitted fabric was fixed to the sample holder of the measurement device. White light of diameter 1 cm was shined on the test piece, and the total light transmittance was measured.
4. Thickness

The thickness of the knitted fabric was measured using a thickness gauge (Mitutoyo No. 2109FL). A 100 mm×100 mm test piece cut out from the knitted fabric was placed between the measurement probes of the thickness gauge and the thickness was measured. The shape of the test probes are half-spheres. The thickness of the joining points and between the joining points were measured.
5. Outer Observation of the Non-engagement Surface

For each embodiment and each comparative example, the outer appearance of the non-engagement surface of the knitted fabric was observed visually.

### (Preparing the Female Material)

### Embodiments 1-6, Comparisons 1-2

The surface fastener female material was prepared according to the conditions in Table 2 and those shown below. The results of evaluation are shown in Table 2.
Knitting machine: 28 gauge 3-bar single tricot knitting machine (made by Karl Mayer)
Yarn arrangement: one-in-one-out
Yarn variety:
84T/36 (decitex/filament) treated yarn (polyester false twist treated yarn) (false twist conditions 4000 tpm (tpm: twist per meter), available commercially through Sanetsu (Corp.))
22T1 untreated yarn (polyester yarn) (available commercially through Toray (Corp.))
56T24 untreated yarn (available commercially through Hyosung Japan (Corp.)) 44T2 untreated yarn (made by doubling 22T1 above as back yarn)

TABLE 2

| | | | | | | | | | Optical | | | | Outer |
|---|---|---|---|---|---|---|---|---|---|---|---|---|---|
| | | | | | | Basis | 135° | MD | Trans- | Thickness (μm) | | | Appearance |
| | | | | | Runner | Weight | Sepa- | Tensile | mit- | Joining | Be- | Aver- | of Non- |
| | Rod | Yarn Type | Weave | Course | (cm) | (g/m²) | ration | Strength | tance | Point | tween | age | engagement |
| Embodiment | F | 84T36 treated | 1-2/0-1/2-1/3-2// | 22C | 130 | 34.8 | 3.21 | 12.1 | 65.8 | 167.2 | 113 | 54.2 | Openings are |
| 1 | M | 22T1 | 1-0/0-1/0-1/I-0// | | 174 | | | | | | | | filled |
| | B | 84T36 treated | 2-3/0-0/3-2/5-5// | | 206 | | | | | | | | |
| Embodiment | F | 84T36 treated | 1-2/0-1/2-1/3-2// | 22C | 160 | 37.5 | 3.46 | 16 4 | 64.1 | 166.2 | 108.2 | 58 | Openings are |
| 2 | M | 22T1 | 1-0/0-1/0-1/1-0// | | 174 | | | | | | | | filled |
| | B | 84T36 treated | 2-3/0-0/3-2/5-5// | | 206 | | | | | | | | |
| Embodiment | F | 84T36 treated | 1-2/0-1/2-1/3-2// | 22C | 200 | 41.2 | 4.68 | 14.0 | 59.9 | 196 | 115.8 | 80.2 | Openings are |
| 3 | M | 22TI | 1-0/0-1/0-1/1-0// | | 174 | | | | | | | | filled |
| | B | 84T36 treated | 2-3/0-0/3-2/5-5// | | 206 | | | | | | | | |
| Embodiment | F | 84T36 treated | 1-2/0-1/2-1/3-2// | 280 | 130 | 43.5 | 3.56 | 9 58 | 59.1 | 162.4 | 119.6 | 42.8 | Openings are |
| 4 | M | 22T1 | 1-0/0-1/0-1/1-0// | | 146 | | | | | | | | filled |
| | B | 84T36 treated | 2-3/0-0/3-2/5-5// | | 206 | | | | | | | | |
| Embodiment | F | 84T36 treated | 1-2/0-1/2-1/3-2// | 28C | 160 | 47 | 3.62 | 11.9 | 57.6 | 172.2 | 117.4 | 54.8 | Openings are |
| 5 | M | 22T1 | 1-0/0-1/0-1/1-0// | | 146 | | | | | | | | filled |
| | B | 84T36 treated | 2-3/0-0/3-2/5-5// | | 206 | | | | | | | | |
| Embodiment | F | 84T36 treated | 1-2/0-1/2-1/3-2// | 28C | 200 | 51.6 | 5.71 | 9 99 | 54.8 | 191.8 | 113.4 | 78.4 | Openings are |
| 6 | M | 22T1 | 1-0/0-1/0-1/1-0// | | 146 | | | | | | | | filled |
| | B | 84T36 treated | 2-3/0-0/3-2/5-5// | | 206 | | | | | | | | |
| Comparative | F | 56T24 | 1-2/0-1/2-1/3-2// | 22C | NA | 21.5 | 0.62 | 26.5 | 88.4 | 103.2 | 22.8 | 80.4 | Many gaps |
| Example 1 | M | 22T1 | 1-0/0-1/0-1/1-0// | | NA | | | | | | | | |
| | B | 44T2 | 2-3/0-0/3-2/5-5// | | NA | | | | | | | | |
| Comparative | F | 56T24 | 1-2/0-1/2-1/3-2// | 22C | NA | 25 | 2.72 | 25.2 | 84.7 | 107.6 | 21 | 86.6 | Many gaps |
| Example 2 | M | 22TI | 1-0/0-1/0-1/I-0// | | NA | | | | | | | | |
| | B | 56T24 | 2-3/0-0/3-2/5-5// | | NA | | | | | | | | |

As shown in Table 2, the female material of embodiments 1-6 with treated yarn as the back yarn had good opacity and smoothness compared to comparative examples 1-2 with untreated yarn as the back yarn, and had excellent engagement strength.

### INDUSTRIAL APPLICABILITY

The surface fastener female material and surface fastener of the present disclosure may be applied particularly favorably in absorbent goods such as hygiene goods, such as, for example, diapers for children, adults, and pets, napkins for hygiene and other uses, and the like.

### REFERENCE NUMERALS

10 surface fastener female material
20 knitted fabric
21, 21F front yarn
22 base weave
22M middle yarn
22B back yarn
1b, 1f, 1m, 2f, 2m, 3b, 3f, 3m, 4f, 4m stitch
2b, 4b insert weave part
a joining point
b intersecting point
30 print part
40 base
41 engagement element
50 male material

1. A surface fastener female material containing a knitted fabric, wherein
the knitted fabric is woven from a back yarn, a middle yarn, and a front yarn,
the back yarn, the middle yarn, and the front yarn alternatingly form a joining point (I) and a joining point (II),
the joining point (I) is composed of a stitch of the back yarn following an overlap in a first direction or a second direction that is opposite the first direction and an underlap in the first direction in front and back of the overlap, a stitch of the front yarn following an overlap in the same direction as the back yarn and an underlap in the same or opposite direction of the back yarn, and a stitch of the middle yarn following an overlap in the direction opposite the back yarn and the front yarn,
the joining point (II) is composed of a stitch of the back yarn following an overlap in the first or the second direction and an underlap in the second direction in front and back of the overlap, a stitch of the front yarn following an overlap in the same direction as the back yarn and the underlap in the same or opposite direction as the back yarn, and a stitch of the middle yarn following the overlap in the opposite direction of the back yarn and the front yarn,
the front yarn forms a close stitch at the joining point (I), the joining point (II), or at both of these, and
the back yarn contains at least 50% treated yarn by the total thread count standard.

2. The surface fastener female material according to claim 1, wherein
the back yarn is knitted having a weave pattern of a stitch forming the joining point (I)/blind lap/stitch forming joining point (II)/blind lap,
the front yarn is knitted having a weave pattern of stitch forming joining point (I)/open lap/stitch forming joining point (II)/open lap, and
the middle yarn is knitted having a chain weave pattern.

3. The surface fastener female material according to claim 1, wherein the front yarn contains at least 50% treated yarn by the total thread count standard.

4. The surface fastener female material according to claim 1, further having a print part.

5. A surface fastener containing the surface fastener female material described in claim 1 and a surface fastener male material.

6. An absorbent good containing the surface fastener described in claim 5.
